(19) United States
(12) Patent Application Publication
(10) Pub. No.: US 2008/0183049 A1
(43) Pub. Date: Jul. 31, 2008

(54) REMOTE MANAGEMENT OF CAPTURED IMAGE SEQUENCE

(75) Inventors: Chris Demetrios Karkanias, Sammamish, WA (US); Stephen E. Hodges, Cambridge (GB); Peter Neupert, Medina, WA (US)

Correspondence Address: AMIN. TUROCY & CALVIN, LLP, 24TH FLOOR, NATIONAL CITY CENTER, 1900 EAST NINTH STREET, CLEVELAND, OH, 44114, US

(73) Assignee: MICROSOFT CORPORATION, Redmond, WA (US)

(21) Appl. No.: 11/669,831

(22) Filed: Jan. 31, 2007

(51) Int. Cl. A61B 5/00

(52) U.S. Cl. 600/301

(57) ABSTRACT

A system that can enable remote monitoring and/or compliance determination by viewing sequences of images captured during an event is disclosed. For example, the innovation can employ captured event sequences to enable a viewer or third party to assess a subject's activities and/or actions. The granularity of the capture of event sequences can be programmed and triggered based upon sensory data. The system also provides mechanisms to locate images or sequences, playback images or sequences or images as well as to set compliance parameters associated with a preference or life style.

### BACKGROUND

As the average life span of our global population increases, there is an ever increasing interest in concern for the elderly. Conventionally, clinicians and health care workers bore the responsibility of understanding the important medical and mental health issues associated with the elderly, while maintaining sensitivity to the social and cultural aspects of this aging population. Developments in technology and science continue to improve the quality of life of the elderly. As well, these developments assist in the treatment of many of the disorders that affect the aging, but there remain many challenges.

‘Elder Care’ can refer to most any service associated with improving the quality of life of the aging population. For example, ‘Elder Care’ can include such services as adult day care, long term care, nursing homes and assisting living facilities, hospice, home care, etc. Within each of these facilities, there is oftentimes a clinician or health care professional on staff, or on call, that locally and personally monitors each of the patients or residents.

Traditionally, care for the elderly was the responsibility of the elder's family. However, because family sizes continue to decrease together with the greater life expectancy of elderly people and the geographic dispersion of families today, Elder Care facilities are on the rise. Another factor leading to the rise in Elder Care facilities is the tendency for women to work outside of the home thus, reducing the amount of family care traditionally available. In general, Elder Care emphasizes the social and personal requirements of older individuals (e.g., senior citizens) who would benefit from assistance with daily activities and health care.

As can be imagined, the cost of Elder Care is also increasing over time due to the overall demand. Whether assisted living or full scale nursing home care is needed, these facilities must be staffed with qualified professionals around the clock. These professionals manually monitor and record criteria associated with the resident which can later be used to evaluate mental and physical condition, rehabilitation progress, exercise regimes, sleep patterns, etc. However, this manual monitoring and recording greatly increases the cost of the care as well as exposure to incorrect diagnostics.

In other words, throughout the resident's daily activity, actions such as exercise, dietary intake, medicinal intake, therapy, rest habits, sleep habits, etc. are most often manually monitored both within a controlled setting as well as throughout daily life. As such, oftentimes, a written or electronic journal is kept that records events such as, how much exercise was done, what was eaten, how much sleep was taken, etc. This manually gathered data is analyzed and recorded in order to determine compliance within the scope of a pre-planned daily routine. However, the self-reporting mechanisms (e.g., written journals and records) are notorious causes of inaccurate data. Moreover, manual supervision and reporting is burdensome and extremely costly to the resident as well as to the Elder Care as a whole.

### SUMMARY

The following presents a simplified summary of the innovation in order to provide a basic understanding of some aspects of the innovation. This summary is not an extensive overview of the innovation. It is not intended to identify key/critical elements of the innovation or to delineate the scope of the innovation. Its sole purpose is to present some concepts of the innovation in a simplified form as a prelude to the more detailed description that is presented later.

The innovation disclosed and claimed herein, in one aspect thereof, comprises a system that can facilitate construction of an electronic journal of a subject's activity within a given time interval or activity (e.g., event). In operation, an event capture component can be given or applied to a subject or patient in order to monitor or journal on-going activity and patterns. Accordingly, family members, health care workers or the like can remotely access the journal to review and/or analyze images.

In a specific example, the journal can be used to determine if a person is adhering to a physical exercise schedule (e.g., physical therapy), keeping with a specific diet, etc. By automatically recording events, the event recorder component can provide a rendition of a day rather than focusing on what was manually written down by a user. This rendition can be used to evaluate standard of living as well as to prompt lifestyle changes where appropriate. Still further, the innovation provides for mechanisms that automatically determine compliance based upon some pre-determined or pre-programmed criteria.

An event recorder component can be used to capture the images associated with events during a wearer's activity. Image capture can be prompted or triggered based upon programmed thresholds, for example, thresholds based upon sensory data (e.g., environmental, physiological), etc. Moreover, information gathered by these sensory mechanisms can also be used to annotate the captured images. These annotations can later be used to assist in locating images or in establishing compliance associated with predetermined criteria.

In addition to determining compliance, aspects of the innovation can manage and/or promote compliance by alerting a subject of a deviation of a compliance parameter. For instance, an alert can be generated and sent to remind a subject to take a take a nap, exercise, eat, etc. These alerts can be audible, visual, vibratory, etc.

To the accomplishment of the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1 illustrates an example system that facilitates employing event image sequences in monitoring a subject (e.g., elderly individual).

FIG. 2 illustrates a block diagram of an example interface component that facilitates monitoring the subject in accordance with an embodiment.

FIG. 3 illustrates an example event management component in accordance with an aspect of the innovation.

FIG. 4 illustrates an example compliance management component in accordance with an aspect of the innovation.

FIG. 5 illustrates a block diagram of an example event recorder component having a sensor component and an event annotation component in accordance with an aspect of the innovation.

FIG. 6 illustrates a block diagram of an event recorder having a physiological sensor component and an environmental sensor component in accordance with an aspect of the innovation.

FIG. 7 illustrates an example compliance management component that facilitates programmatically establishing trigger and compliance criteria in accordance with an aspect of the innovation.

FIG. 8 illustrates an architecture including a machine learning and reasoning component that can automate functionality in accordance with an aspect of the innovation.

FIG. 9 illustrates an exemplary flow chart of procedures that facilitate compliance determination via viewing image sequences of event activity in accordance with an aspect of the innovation.

FIG. 10 illustrates an exemplary flow chart of procedures that facilitate annotating image sequences with context data (e.g., physiological, environmental) in accordance with an aspect of the innovation.

FIG. 11 illustrates an exemplary flow chart of procedures that facilitates employing annotations to enhance playback of captured images in accordance with an aspect of the innovation.

FIG. 12 illustrates a block diagram of a computer operable to execute the disclosed architecture.

FIG. 13 illustrates a schematic block diagram of an exemplary computing environment in accordance with the subject innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

As used herein, the term to “infer” or “inference” refer generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

The subject innovation is directed to systems and methods that enable viewers to remotely access images or sequences of images captured by a camera which monitors an individual's activity. More particularly, an image capture device can be worn, attached or applied to a user such that it can establish a journal of actions and activities within a period of time (e.g., day). Accordingly, the subject innovation enables remote access to the captured images in order to enable monitoring and/or assessment of a subject user.

Still further, in other aspects, the innovation enables triggers and compliance criteria to be programmed. The trigger criteria can define when images are to be captured. For example, images can be captured in response to a change in environmental (e.g., location, lighting, temperature) or physiological (e.g., heart rate, blood pressure, body temperature) factors, etc. Compliance criteria can be used to monitor and assess a subject's activity such as, dietary intake, sleep habits, exercise routine, etc. Effectively, the system can automatically analyze the captured image sequences thereafter comparing them to a predetermined compliance criteria to reach a compliance determination.

Although most of the aspects described herein are directed to care of the elderly, it is to be appreciated that the innovation can be employed to monitor most any individual. For example, the systems described herein can be employed to monitor a child in daycare where it may be desirable to monitor a particular amount or type of food intake, physical activity, sleep amounts, etc.

Essentially, the innovation discloses systems and/or methods that can employ the captured information to establish compliance as a function of some predetermined criteria. As well, systems and methods can automatically regulate compliance, for example, by delivering notifications in the event of a compliance deviation. In doing so, captured information can be analyzed to determine if a subject is adhering to criteria associated with the predetermined criteria and, if not, suggestions (via notifications) can be made to prompt adherence. In aspects, notifications can be of the form of an email, an instant message, an audio or visual prompt, etc.

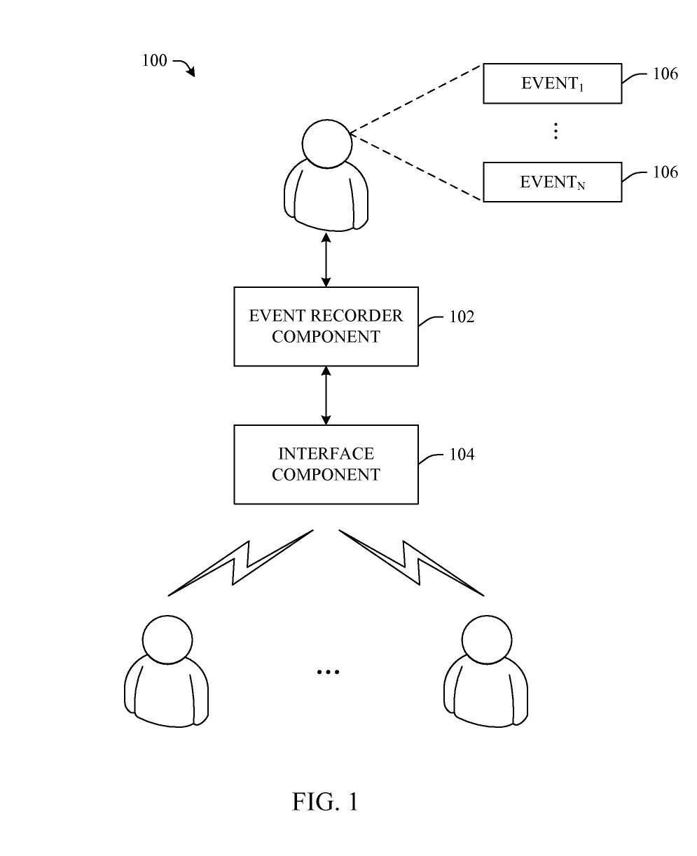

Referring initially to the drawings, FIG. 1 illustrates a system 100 that facilitates remote management and access to images captured in accordance with a subject's actions and/or activities. These captured images and sequences of images can be used to remotely monitor and determine compliance in accordance with predefined criteria. Generally, system 100 can include an event recorder component 102 that captures images and an interface component 104 that enables a user to remotely interact with the event recorder component 102.

More particularly, the event recorder component 102 can be used to capture images and sequences of images that establish a visual journal of a subject's activity within a defined time period (e.g., hour, day, month). As will be better understood upon a review of the figures that follow, the triggers can be programmed that prompt capture of images and other descriptive information thereby facilitating establishment of the visual journal. The interface component 104 enables a viewer or other third party to gain access to images via the event recorder component 102.

In operation, access can be gained remotely via any suitable wired or wireless network (e.g., 802.11). In other words, a viewer can access archived images as well as real-time images via the interface component 104. As illustrated in FIG. 1, the event recorder component 102 can be used to capture images of 1 to N events, where N is an integer. It is to be understood that 1 to N events can be referred to individually or collectively as events 106. By way of example, events can be a specific activity (e.g., meal, sleep, exercise) or any combination of activities. For instance, an event 106 might be defined by a period of time, e.g., Friday, whereas the event recorder component 102 can be used to capture details associated with activity within the defined event 106.

In other aspects, the system 100 illustrated in FIG. 1 can be used in conjunction with a remote call device (e.g., emergency pendant) which can be manually triggered by the wearer. For example, a user can also wear an emergency pendant (or can be integrated into system 100) such that, when activated, image capture can be triggered and communication can be made automatically with some remote entity (e.g., call center, health care facility). This communication can automatically prompt remote access with the event recorder component 102 or stored images as desired. Moreover, it is to be understood that access to images captured via the event recorder component can refer to real-time access as well as access to stored images. These stored images can be located within a common storage device (e.g., hard disk, memory, cache, buffer), distributed storage device or combination thereof.

Furthermore, although the figures included herewith illustrate components associated with particular systems, it is to be understood that these components can be selectively distributed within the system or other network without departing from the spirit and/or scope of the innovation. For example, while many of the elements can be run within a wearable device (e.g., event recorder component), it is to be understood that, alternatively, all or a subset of the components can be located on a client device used to remotely access images and information. Still further, in other aspects, some components can be located between the event recorder component 102 and the client device, for example on servers, on the Internet, within a cloud, etc. without departing from the spirit and scope of this specification and claims appended hereto.

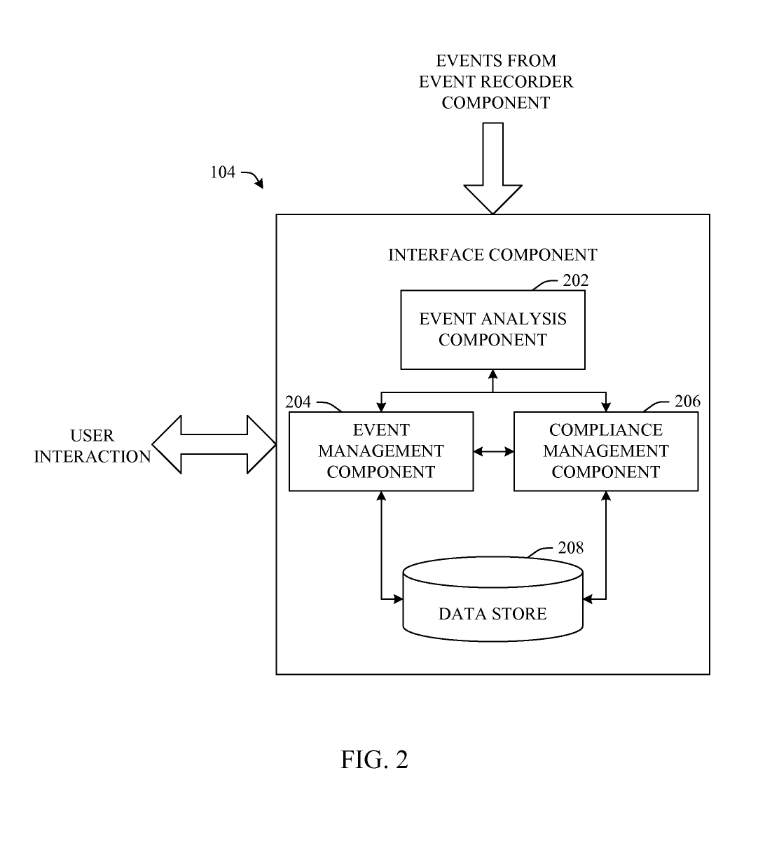

Referring now to FIG. 2, a block diagram of an example interface component 104 is shown. As illustrated, the interface component 104 can include an event analysis component 202, an event management component 204, a compliance management component 206 and a data store 208. Each of these components (202, 204, 206) enable a viewer or third party (e.g., family member, clinician, health care professional) to remotely monitor activity of a subject. As described above, this monitoring can occur in real-time as well as retroactively after a visual journal has been established.

Although many of the aspects described herein address automatic compliance determination, it is to be understood that the compliance management component 206 is optional to the innovation described herein. As such, it is to be understood that, in a simple case, the innovation enables a viewer or third-party to access and/view images captured via an event recorder component 102. Thus, a viewer can use the images as preferred and/or desired (e.g., to determine compliance, adjust lifestyle of subject).

The event analysis component 202 can be employed to interpret captured images related to a specified event 106. This interpretation can be used by the event management component 204 and/or the compliance management component 206 to search/view images as well as to reach a compliance determination respectively. Each of these components (204, 206) will be described in greater detail with respect to the figures that follow. The data store 208 can be used to store captured images as well as trigger and compliance criteria as desired.

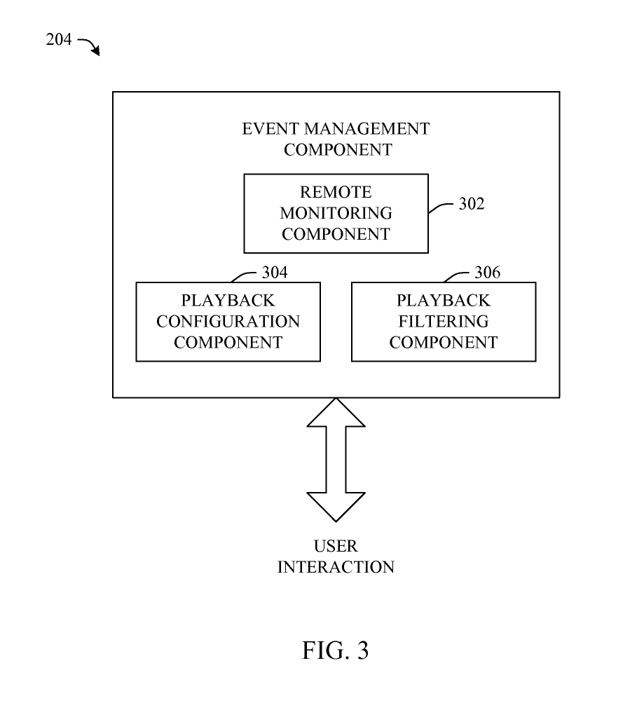

FIG. 3 illustrates a block diagram of an example event management component 204. As shown, the event management component 204 can include a remote monitoring component 302, a playback configuration component 304 and/or a playback filtering component 306. In operation, the subcomponents (302, 304, 306) shown in FIG. 3 facilitate remote monitoring (and access) to images and sequences of images.

The remote monitoring component 302 provides a gateway for access to images of the event recorder component 102. As described above, the remote monitoring component 302 can facilitate access to real-time images as well as images that are captured and stored in the form of an event sequence. It is to be appreciated that suitable authentication and/or authorization techniques can be employed to maintain privacy and/or confidentiality of the images.

In one example, an event recorder component 102 can be employed to monitor actions and activities of an elderly person. As such, the remote monitoring component 302 can be employed by approved family member, clinicians, health care professionals or the like in order to monitor the actions and/or activities of the elderly subject. For instance, the remote monitoring component 302 can be used to monitor dietary intake, exercise routines, sleep amounts and habits, etc. Still further, as will be described below, the system can monitor environmental factors (e.g., temperature, location, time of day, external noises) as well as physiological factors (e.g., heart rate, blood pressure, body temperature). These environmental and/or physiological factors can be used to better interpret images and/or sequences of images captured via the event recorder component 102.

The playback configuration component 304 can be employed to manage playback of images captured via the event recorder component. It is to be understood that, although each of these components are shown inclusive of the event management component 204, it is to be understood that each of the components (302, 304, 306) can be employed independent of the others without departing from the spirit and/or scope of this disclosure and claims appended hereto.

In operation, the playback configuration component 304 can be employed to visually review images of events 106 captured via the event recorder component (102 of FIG. 1). The playback filtering component 306 provides a mechanism whereby a user (e.g., third party) can search for and retrieve images related to a subject's activity. Essentially, the event management component 302 provides mechanisms whereby a viewer can remotely interact with the images captured via the event recorder component 102.

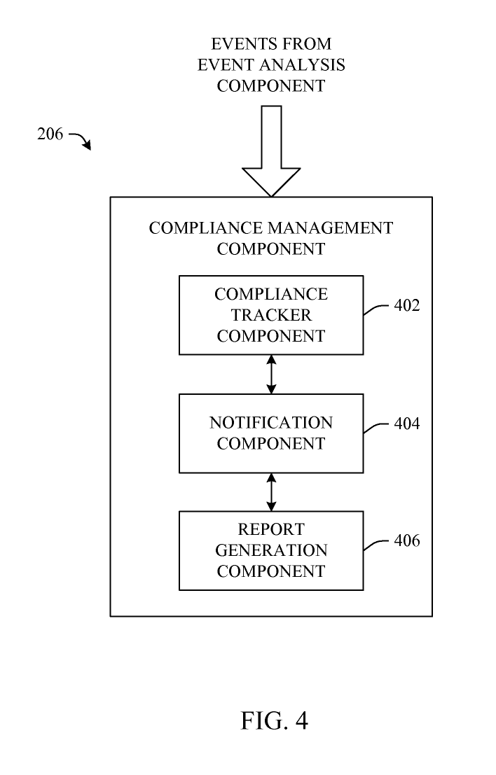

As described with respect to FIG. 2 supra, it is to be understood and appreciated that the interface component 104 could also be equipped with a compliance management component 206. As shown in FIG. 4, the compliance management component 206 can include a compliance tracker component 402, a notification component 404 and/or a report generation component 406. Additionally, as will be described infra, the compliance management component 206 can also include mechanisms that enable criteria to be programmed such as, triggering and compliance criteria. These criteria can be used by a monitoring entity to determine compliance with a pre-defined or pre-planned routine or regime.

In operation, the compliance tracker component 402 can facilitate management and/or monitoring of compliance based upon predetermined criteria. In operation, the event recorder component 102 can automatically record images of related to events 106 associated with the actions of an individual within a given period of time or associated with a selected function or activity.

Further, the event recorder component 102 can be employed to capture images related to actions of a user within some pre-defined period of time or activity. The granularity and frequency of the captured events can be programmed, pre-programmed or contextually triggered via the event recorder component 102. By way of example, the event recorder component 102 can be equipped with sensors (e.g., light sensors, location sensors, motion sensors) whereby when a change in a designated criterion is detected, an image of the event 106 is captured. As will be described below, the compliance management component 206 can be equipped with mechanisms by which these triggering criteria can be programmed.

The compliance tracker component 402 can be employed to automatically monitor captured images in order to determine compliance as a function of compliance criteria. For example, the images can be analyzed to verify dietary intake, awake/sleep times, exercise routine(s), etc. Additionally, physiological and/or environmental data associated with the subject can be captured and subsequently used to tag or annotate an image sequence. This physiological and/or environmental data can assist in establishing compliance with predetermined criteria as well as to assist in playback of captured images.

The notification component 404 and report generation component 406 can also be used to assist in monitoring and/or compliance regulation. For instance, the notification component 404 can be employed to prompt or generate an alarm to the wearer and/or monitoring entity of a deviation in compliance. The notification or alarm can take most any form desired including, but not limited to a basic audible, visual or vibratory notification, an email, an instant message, an SMS (short message service) message or the like.

The report generation component 406 can be employed to quantify and/or render information to an observer of a subject. For example, a report can be generated and rendered via a display to identify specifics related to dietary intake, exercise, etc. It is to be understood that the report generation component 406 can be employed to render information related to the subject as desired. This information can be information gathered and retrieved via the image capture functionality as well as the sensory mechanisms employed.

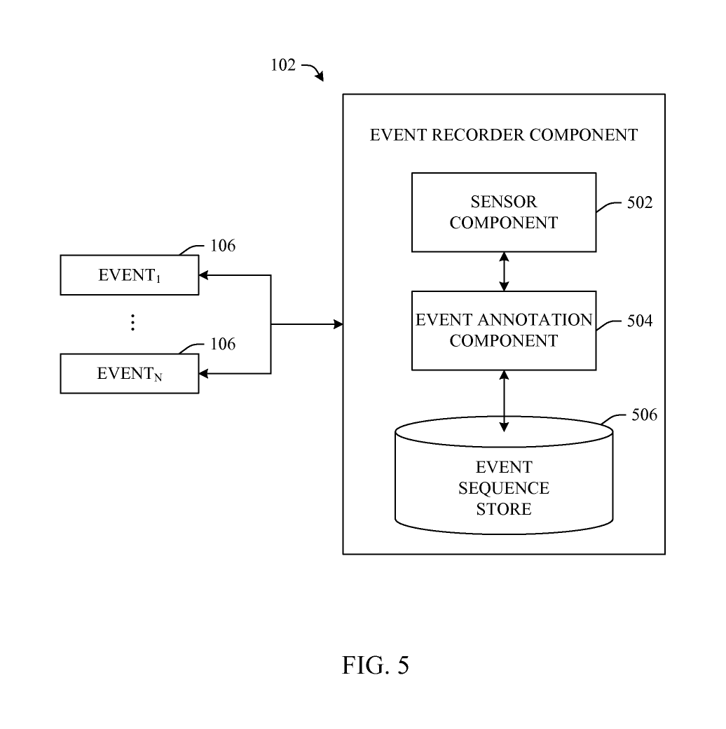

Turning now to FIG. 5, a block diagram of an example event recorder component 102 is shown. In general, in addition to the image capture functionality, event recorder component 102 can include a sensor component 502, an event annotation component 504 and an event sequence store 506. It is to be understood and appreciated that the event recorder component 102 can be a wearable image capture device (e.g., camera) that establishes a digital record of the events 106 that a person experiences. This digital record can be maintained within the event sequence store 506 for later analysis and/or playback. The nature of the device (102) is to capture these recordings automatically, without any user intervention and therefore without any conscious effort. However, image capture can also be user-initiated in other aspects.

As described supra, one rationale of the event recorder component 102 is that a captured digital record of an event 106 can subsequently be reviewed in order to determine compliance of and to assist with monitoring elderly individuals (e.g., via compliance tracker component 402). As well, review of the captured images can be used to assist in prompting, notifying or alerting a subject of a deviation of a predefined parameter.

As illustrated, the event recorder component 102 can include a sensor component 502 and an optional event annotation component 504 which facilitate prompting action and index with regard to the images. Essentially, the sensor component 502 can be used to identify information that triggers the capture of an image from the event recorder component 102.

The optional event annotation component 504 can facilitate annotating (or tagging) image sequences. As described above, sensor data can be captured and used to annotate an image sequence to assist in comprehensive utilization of the captured images. For instance, the annotations can be applied in the form of metadata and employed to assist in enhancing playback of the captured images.

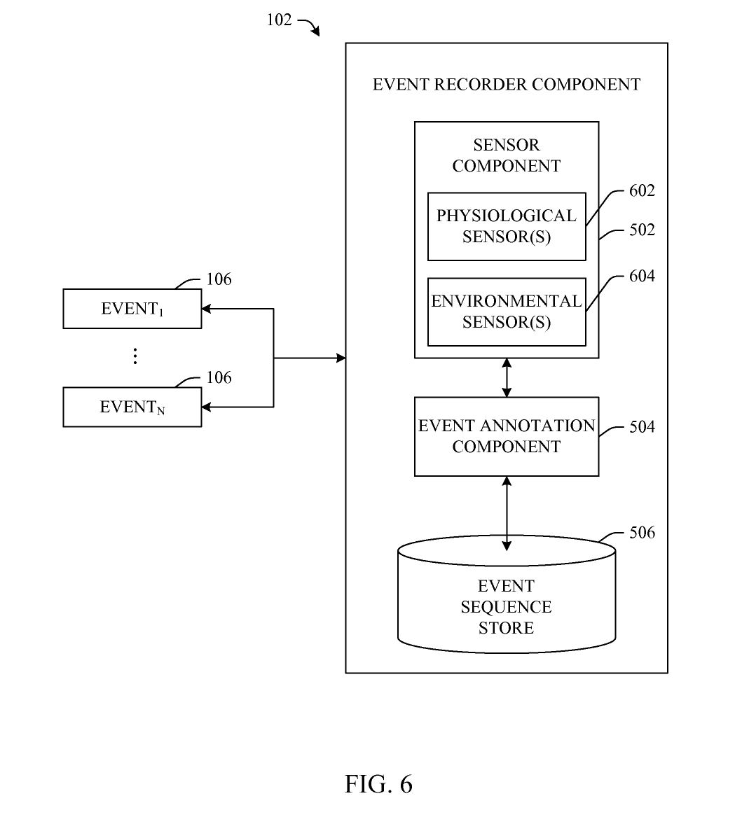

Referring now to FIG. 6, the sensor component 502 can include either or both physiological and/or environmental sensors (602, 604). In operation, these sensors can be used to trigger image capture as well as to gather information and data to be used in annotating images. For instance, when a specific threshold is reached, an image or series of images can be automatically captured and annotated with the data related to the triggering threshold. Similarly, when an image is captured, environmental and/or physiological data can be simultaneously captured and employed to annotate captured images.

By way of example, the event recorder component 102 can automatically capture an image of an event 106, for example, an image of a subject exercising or resting. In addition to capturing the image of the event 106, the event recorder component 102 (via sensors 602, 604) can monitor physiological criterion such as heart rate, blood pressure, body temperature, blood sugar, blood/alcohol concentration, etc. associated with the event 106. As well, environmental data such as location, ambient temperature, weather conditions, etc. can be captured. Thus, this annotated data related to the event 106 can be used to more intelligently assess the compliance with predetermined criteria.

At a low level, the image recorder component can be employed to capture image sequences related to events 106 (e.g., exercise sessions, rest periods, meal times). This information can be used to ensure compliance, for example amount of exercise or rest, timing and nutritional value of meals, etc. Additionally, environmental data (e.g., ambient temperature, location, motion) can be captured to assist in analysis of events 106 related to desired criterion. Moreover, physiological data can be captured and employed to further assist in analysis of events 106 associated with the predefined criteria.

In other aspects, the event recorder component 102 can effectively be employed as an information hub for a viewer to monitor a subject. For example, it is to be appreciated that a series of specialized sensors 502 that integrate with the event recorder component 102, for example, via some wireless link (e.g., Bluetooth, infrared, IEEE 802.11, cell network) can be employed. In this way, event recorder component 102 could have a general core of data collection (e.g., global position system data (GPS), image data, temperature data, audio data, motion data, identification gathered data) but could be adapted to specific measures with what, in effect, could be modular ‘add-ons.’ By way of further example, vital sign sensors, a pulse-oximeter, a stretch or range-of-motion sensor, skin galvanic response measuring sensor, or a gait sensor (pressure sensitive insert in your shoes) could be employed as modular ‘add-ons’ to the core event recorder component 102. Thus, the event recorder component 102 can be employed as an overall information hub for information and data related to a monitored subject.

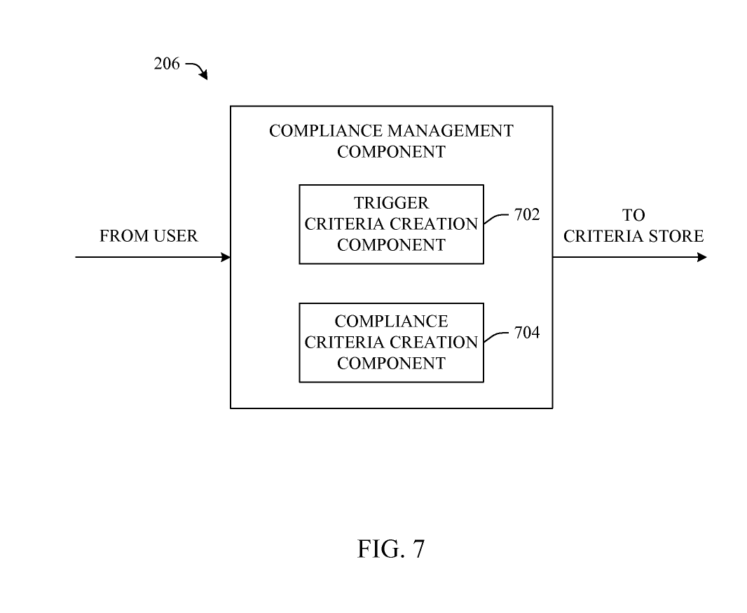

Referring now to FIG. 7, a block diagram of compliance management component 206 is shown. As illustrated, the compliance management component 206 can include a trigger criteria creation component 702 and a compliance criteria creation component 704. Each of these components (702, 704) facilitates a viewer or monitoring entity to programmatically set criteria that prompts image and information capture as well as compliance determination. In other words, thresholds that prompt when the event recorder component captures images and information can be set via the trigger criteria creation component 702. Similarly, compliance thresholds can be set via the compliance criteria creation component 704.

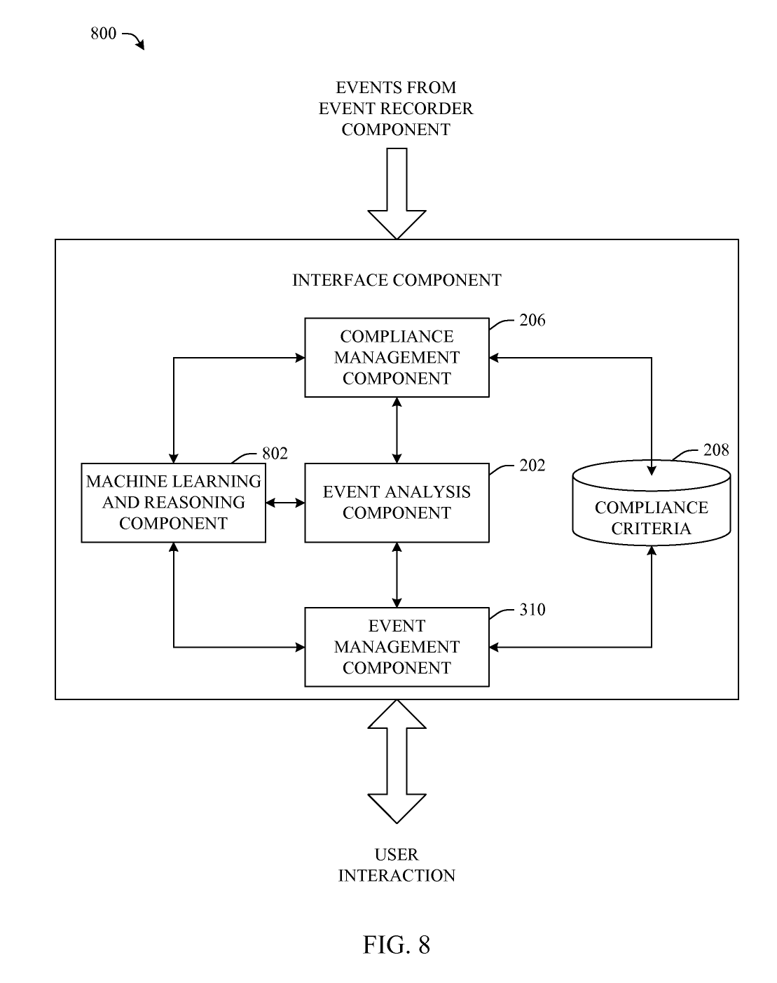

FIG. 8 illustrates a system 800 that employs machine learning and reasoning (MLR) component 802 which facilitates automating one or more features in accordance with the subject innovation. The subject innovation (e.g., in connection with prompting image capture, establishing compliance, notification) can employ various MLR-based schemes for carrying out various aspects thereof. For example, a process for determining when to trigger the event recorder component 102 to begin capture can be facilitated via an automatic classifier system and process. Moreover, MLR techniques can be employed to automatically establish compliance criteria, assess compliance, etc.

A classifier is a function that maps an input attribute vector, x=(x1, x2, x3, x4, xn), to a confidence that the input belongs to a class, that is, f(x)=confidence(class). Such classification can employ a probabilistic and/or statistical-based analysis (e.g., factoring into the analysis utilities and costs) to prognose or infer an action that a user desires to be automatically performed.

A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated from the subject specification, the subject innovation can employ classifiers that are explicitly trained (e.g., via a generic training data) as well as implicitly trained (e.g., via observing user behavior, receiving extrinsic information). For example, SVM's are configured via a learning or training phase within a classifier constructor and feature selection module. Thus, the classifier(s) can be used to automatically learn and perform a number of functions, including but not limited to determining according to a predetermined criteria when to trigger capture of an image, how/if to annotate an image, what thresholds should be set for compliance, what granularity to capture images (e.g., number of frames per second), etc.

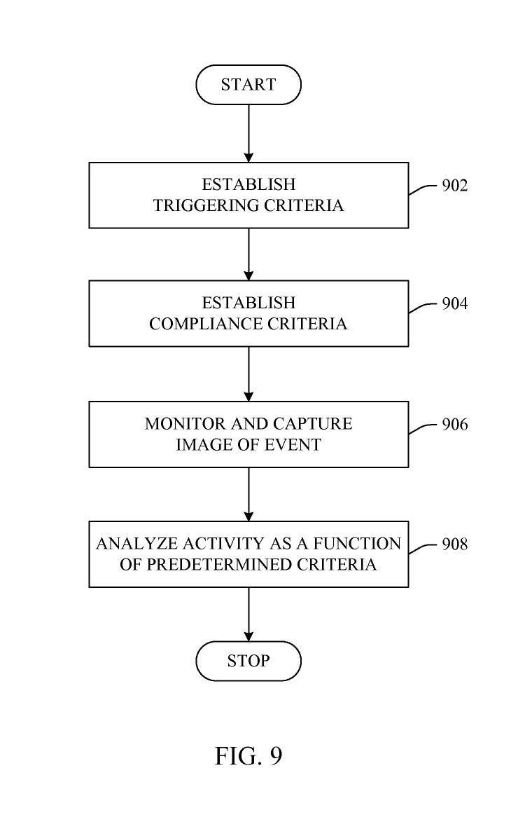

FIG. 9 illustrates a methodology of employing a sequence of event images in order to monitor a subject in accordance with an aspect of the innovation. While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

At 902 and 904, triggering and compliance criteria can be established. For example, triggering criterion can be established that controls when/if an image is captured. As well, the triggering criteria can control sensory technologies with regard to when/if environmental and physiological information is captured and gathered. The compliance criteria can be established in order to identify thresholds related to desired criteria. For instance, a threshold can be established that relates to dietary intake, exercise routines, sleep patterns, etc. As will be described below, these criterion can be used to enhance capture and compliance of information related to a subject.

At 906, events can be monitored and images of the events captured. In examples, an event can span a specified period of time or an interval within a period of time. Still further, an event can be defined by a specific action of a user or subject. For instance, an event can be a visit to the park or an exercise session.

Continuing at 906, image sequences of events are captured. As described above, the granularity of the capture of images can be based upon the scope of the monitoring. Thus, the granularity can be preprogrammed or inferred (e.g., via MLR) based upon information related to the subject, including but not limited to demographic information, age, health/mental condition, context, activity, etc. It will further be understood that the image capture can be triggered based upon information gathered via the environmental and/or physiological sensors.

At 908, the captured images can be employed to analyze activity as a function of the compliance criteria. In other words, a monitoring entity, viewer, auditor or other third party can view the images in order to determine compliance with the predetermined criterion. It is to be understood that the analysis can occur after all events are monitored or could possibly occur in real-time. For example, intelligence could be employed to automatically perform analysis during (as well as immediately following) an event. Similarly, a link (e.g., wireless link) could be employed to upload images to enable remote analysis either automatically and/or manually as desired.

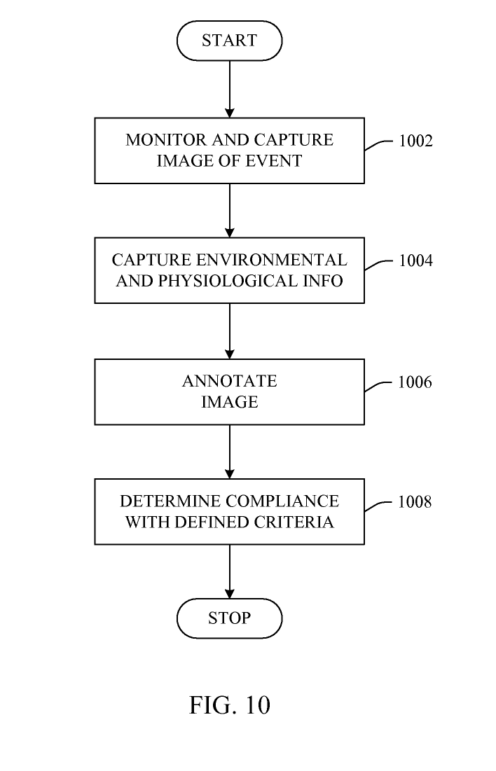

Referring now to FIG. 10, there is illustrated a methodology of automatically determining compliance against predetermined criteria by annotating images in accordance with the innovation. Specifically, at 1002, subject activity and action can be monitored. For instance, the system can monitor actions as they relate to eating, exercise, rest, communication, etc.

Images related to an event (or sequence of events) can be captured at 1002. As described above with reference to FIG. 9, capture of these images can be triggered as a function of the sensor data, preprogrammed interval data, etc. For example, a proximity sensor can be employed to trigger image capture when a subject is within a certain distance of an RFID (radio frequency identification) equipped location or object. Additionally, as described supra, at 1004, external data related to an event can be captured via physiological and/or environmental sensors.

Once captured, the images and/or sequences of images can be annotated with contextual data (and other sensor-provided data) at 1006. These annotations can provide additional data to assist in determination and effects within the scope of monitoring a subject. At 1008, the annotated images can be employed to determine compliance with regard to preprogrammed parameters and/or thresholds.

Although both FIG. 9 and FIG. 10 are illustrated in the form of a linear flow diagram, it is to be understood that the acts described can be performed recursively in accordance with additional events or portions thereof. As well, it is to be understood that analysis and/or compliance determination need not occur after all images are captured. Rather, analysis and/or compliance can be determined at any time (e.g., in real-time) as desired.

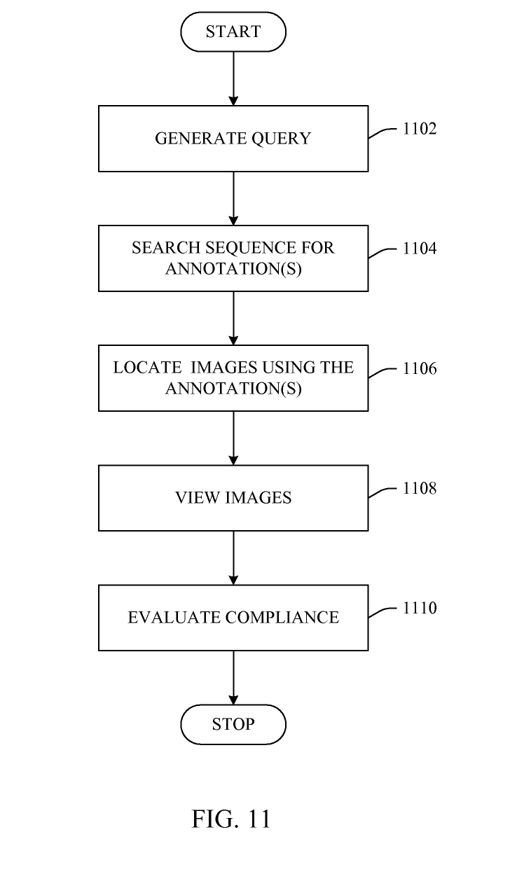

With reference now to FIG. 11, a methodology of searching for a specific event(s) and employing the events to evaluate compliance in accordance with the innovation is shown. Initially, at 1102, search parameters (e.g., query) can be generated. For example, search criteria can be configured to locate images that correspond to specific instances of exercise or rest as desired.

A search can be conducted at 1104 in order to locate desired images and/or sequences of images. In aspects, pattern and audio recognition mechanisms can be employed in order to search for and locate desired images and/or sequences that match a defined query. Similarly, these pattern and/or audio recognition systems can be employed to pre-annotate images thereafter effectuating the search and subsequent retrieval at 1106.

Once retrieved, the images can be viewed at 1108 to assist in determining compliance at 1110. Essentially, sensor data (e.g., visual journal of sensor data) related to the use of a medication and/or treatment can be employed to determine compliance with some desired criterion. Additionally, this visual journal can be searchable based upon content or other annotations (e.g., environmental data, physiological data).

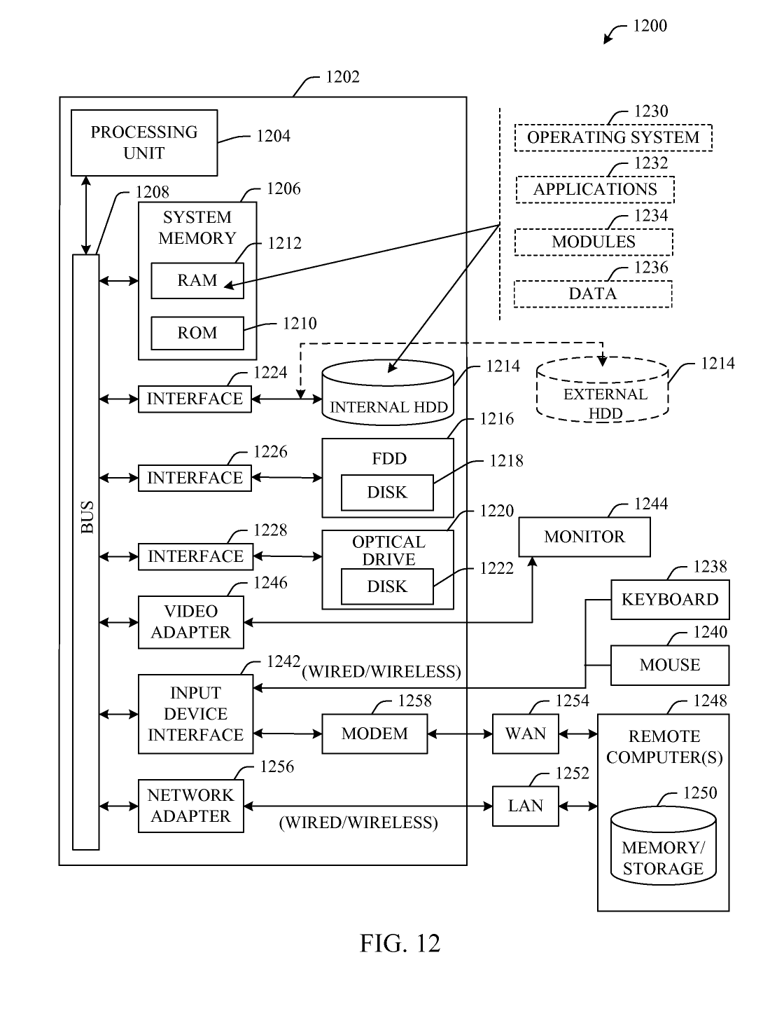

Referring now to FIG. 12, there is illustrated a block diagram of a computer operable to execute the disclosed architecture. In order to provide additional context for various aspects of the subject innovation, FIG. 12 and the following discussion are intended to provide a brief, general description of a suitable computing environment 1200 in which the various aspects of the innovation can be implemented. While the innovation has been described above in the general context of computer-executable instructions that may run on one or more computers, those skilled in the art will recognize that the innovation also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

With reference again to FIG. 12, the exemplary environment 1200 for implementing various aspects of the innovation includes a computer 1202, the computer 1202 including a processing unit 1204, a system memory 1206 and a system bus 1208. The system bus 1208 couples system components including, but not limited to, the system memory 1206 to the processing unit 1204. The processing unit 1204 can be any of various commercially available processors. Dual microprocessors and other multi-processor architectures may also be employed as the processing unit 1204.

The system bus 1208 can be any of several types of bus structure that may further interconnect to a memory bus (with or without a memory controller), a peripheral bus, and a local bus using any of a variety of commercially available bus architectures. The system memory 1206 includes read-only memory (ROM) 1210 and random access memory (RAM) 1212. A basic input/output system (BIOS) is stored in a non-volatile memory 1210 such as ROM, EPROM, EEPROM, which BIOS contains the basic routines that help to transfer information between elements within the computer 1202, such as during start-up. The RAM 1212 can also include a high-speed RAM such as static RAM for caching data.

The computer 1202 further includes an internal hard disk drive (HDD) 1214 (e.g., EIDE, SATA), which internal hard disk drive 1214 may also be configured for external use in a suitable chassis (not shown), a magnetic floppy disk drive (FDD) 1216, (e.g., to read from or write to a removable diskette 1218) and an optical disk drive 1220, (e.g., reading a CD-ROM disk 1222 or, to read from or write to other high capacity optical media such as the DVD). The hard disk drive 1214, magnetic disk drive 1216 and optical disk drive 1220 can be connected to the system bus 1208 by a hard disk drive interface 1224, a magnetic disk drive interface 1226 and an optical drive interface 1228, respectively. The interface 1224 for external drive implementations includes at least one or both of Universal Serial Bus (USB) and IEEE 1394 interface technologies. Other external drive connection technologies are within contemplation of the subject innovation.

The drives and their associated computer-readable media provide nonvolatile storage of data, data structures, computer-executable instructions, and so forth. For the computer 1202, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the innovation.

A number of program modules can be stored in the drives and RAM 1212, including an operating system 1230, one or more application programs 1232, other program modules 1234 and program data 1236. All or portions of the operating system, applications, modules, and/or data can also be cached in the RAM 1212. It is appreciated that the innovation can be implemented with various commercially available operating systems or combinations of operating systems.

A user can enter commands and information into the computer 1202 through one or more wired/wireless input devices, e.g., a keyboard 1238 and a pointing device, such as a mouse 1240. Other input devices (not shown) may include a microphone, an IR remote control, a joystick, a game pad, a stylus pen, touch screen, or the like. These and other input devices are often connected to the processing unit 1204 through an input device interface 1242 that is coupled to the system bus 1208, but can be connected by other interfaces, such as a parallel port, an IEEE 1394 serial port, a game port, a USB port, an IR interface, etc.

A monitor 1244 or other type of display device is also connected to the system bus 1208 via an interface, such as a video adapter 1246. In addition to the monitor 1244, a computer typically includes other peripheral output devices (not shown), such as speakers, printers, etc.

The computer 1202 may operate in a networked environment using logical connections via wired and/or wireless communications to one or more remote computers, such as a remote computer(s) 1248. The remote computer(s) 1248 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1130 is illustrated. The logical connections depicted include wired/wireless connectivity to a local area network (LAN) 1132 and/or larger networks, e.g. a wide area network (WAN) 1134. Such LAN and WAN networking environments are commonplace in offices and companies, and facilitate enterprise-wide computer networks, such as intranets, all of which may connect to a global communications network, e.g., the Internet.

When used in a LAN networking environment, the computer 1202 is connected to the local network 1132 through a wired and/or wireless communication network interface or adapter 1136. The adapter 1136 may facilitate wired or wireless communication to the LAN 1132, which may also include a wireless access point disposed thereon for communicating with the wireless adapter 1136.

When used in a WAN networking environment, the computer 1202 can include a modem 1138, or is connected to a communications server on the WAN 1134, or has other means for establishing communications over the WAN 1134, such as by way of the Internet. The modem 1138, which can be internal or external and a wired or wireless device, is connected to the system bus 1208 via the serial port interface 1242. In a networked environment, program modules depicted relative to the computer 1202, or portions thereof, can be stored in the remote memory/storage device 1130. It will be appreciated that the network connections shown are exemplary and other means of establishing a communications link between the computers can be used.

The computer 1202 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices.

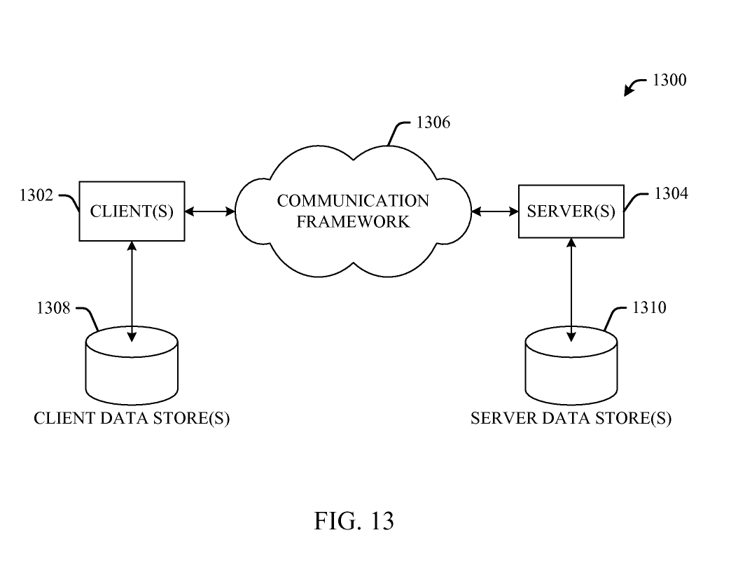

Referring now to FIG. 13, there is illustrated a schematic block diagram of an exemplary computing environment 1300 in accordance with the subject innovation. The system 1300 includes one or more client(s) 1302. The client(s) 1302 can be hardware and/or software (e.g., threads, processes, computing devices). The client(s) 1302 can house cookie(s) and/or associated contextual information by employing the innovation, for example.

The system 1300 also includes one or more server(s) 1304. The server(s) 1304 can also be hardware and/or software (e.g., threads, processes, computing devices). The servers 1304 can house threads to perform transformations by employing the innovation, for example. One possible communication between a client 1302 and a server 1304 can be in the form of a data packet adapted to be transmitted between two or more computer processes. The data packet may include a cookie and/or associated contextual information, for example. The system 1300 includes a communication framework 1306 (e.g., a global communication network such as the Internet) that can be employed to facilitate communications between the client(s) 1302 and the server(s) 1304.

Communications can be facilitated via a wired (including optical fiber) and/or wireless technology. The client(s) 1302 are operatively connected to one or more client data store(s) 1308 that can be employed to store information local to the client(s) 1302 (e.g., cookie(s) and/or associated contextual information). Similarly, the server(s) 1304 are operatively connected to one or more server data store(s) 1310 that can be employed to store information local to the servers 1304.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system that facilitates remote monitoring of an individual, comprising:
an event recorder component that captures a plurality of images associated with an event, the event represents an interval in the individual's activity; and
an interface component that enables a viewer to remotely access a subset of the images associated with the event.

2. The system of claim 1, further comprising an event management component that facilitates remote access to the subset of the plurality of the images.

3. The system of claim 2, further comprising a remote monitoring component that facilitates the viewer to monitor the events in real-time.

4. The system of claim 1, further comprising a playback configuration component that facilitates configuration of the subset of the images in accordance with a user preference.

5. The system of claim 1, further comprising a playback filtering component that facilitates selection of the subset of images.

6. The system of claim 1, further comprising a compliance management component that facilitates establishing compliance criteria and comparing the subset of the images to the compliance criteria to make a compliance determination.

7. The system of claim 6, further comprising a compliance tracker component that automatically tracks compliance of the event as a function of the subset of the images in view of the compliance criteria.

8. The system of claim 7, further comprising a notification component that generates an alert that advises a viewer of a deviation by the individual as a function of the compliance criteria.

9. The system of claim 7, further comprising a report generation component that generates a report that identifies the compliance as a function of the compliance criteria.

10. The system of claim 1, further comprising an event analysis component that dynamically analyzes the subset of images as a function of third-party generated compliance criteria and establishes a compliance determination based upon the analysis.

11. The system of claim 1, further comprising a sensor component that gathers information that triggers image capture via the event recorder component.

12. The system of claim 11, the sensor component includes at least one of a physiological and an environmental sensor component.

13. The system of claim 11, further comprising an event annotation component that employs information to annotate the subset of the images; the annotation facilitates compliance determination via analysis of the subset of the images.

14. The system of claim 11, the information includes at least one of activity information, medication information, exercise information, time/date information, environmental data or physiological data.

15. The system of claim 1, further comprising a trigger criteria creation component that facilitates establishment of triggering criteria which is employed to prompt capture of the plurality of images.

16. The system of claim 1, further comprising a compliance criteria creation component that enables establishment of the compliance criteria.

17. A method of monitoring activity of an individual, comprising:
capturing a sequence of images associated with an action of the individual;
remotely accessing a subset of the images associated with the action; and
determining compliance of the action represented within the subset of images as a function of a predefined compliance criterion.

18. The method of claim 17, further comprising:
establishing triggering criteria which prompts capture of the sequence of images; and
establishing the compliance criterion used to determine compliance as a function of the subset of the images.

19. The method of claim 17, further comprising:
defining a querying of an event sequence store;
locating the subset of images from the event sequence store as a function of the query; and
retrieving the subset of images from the event sequence store.

20. A system that facilitates remote access to captured images associated to a wearer of an event recorder, comprising:
means for establishing triggering criteria;
means for establishing compliance criteria;
means for capturing a sequence of images associated with an event based upon the triggering criteria;
means for annotating images with information associated with at least one of identity of the user, an environmental condition, a physiological condition, a date or a time of day;
means for remotely accessing a subset of the annotated images; and
means for automatically establishing a compliance determination as a function of content of the annotated subset of images in view of the compliance criterion.
